SEQUENCE LISTING (1 sequence)

SEQ ID NO: 1 - DNA, 1517 bp, Lactobacillus sp.
gcgtgcctaatacatgcaagtcgaacgaaacttctttatcaccgagtgcttgcactcaccgataaagagttgagtggcgaacgggtgagtaacacgtgggcaacctgcccaaaagagggggataacacttggaaacaggtgctaataccgcataaccatagttaccgcatggtaactatgtaaaaggtggctatgctaccgcttttggatgggcccgcggcgcattagctagttggtggggtaaaggcttaccaaggcaatgatgcgtagccgaactgagaggttgatcggccacattgggactgagacacggcccaaactcctacgggaggcagcagtagggaatcttccacaatgggcgaaagcctgatggagcaacgccgcgtgggtgaagaaggtcttcggatcgtaaaaccctgttgttagagaagaaagtgcgtgagagtaactgttcacgtttcgacggtatctaaccagaaagccacggctaactacgtgccagcagccgcggtaatacgtaggtggcaagcgttatccggatttattgggcgtaaagggaacgcaggcggtcttttaagtctgatgtgaaagccttcggcttaaccggagtagtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaagtgttggagggtttccgcccttcagtgctgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcttttgccaatcctagagataggactttcccttcggggacaaaatgacaggtggtgcatggttgtcgtcagctcgtgtcgtgagatgttgggttaagtcccgcaacgagcgcaacccttattgttagttgccagcattaagttgggcactctagcaagactgccggtgacaaaccggaggaaggtggggatgacgtcaaatcatcatgccccttatgatctgggctacacacgtgctacaatggacggtacaacgagtcgcaagaccgcgaggtttagcaaatctcttaaagccgttctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtggggtaaccttttggagccagccgtctaaggtgggacagatgattagggtgaagtcgtaacaaggtagccgtaggagaacctgcggctggatcacc

(19) United States
(12) Patent Application Publication
(10) Pub. No.: US 2022/0162547 A1
(43) Pub. Date: May 26, 2022

(54) TREATMENT OF RESPIRATORY DISEASES WITH A BACTERIUM OF THE GENUS LACTOBACILLUS

(71) Applicant: Université Paul Sabatier Toulouse III, TOULOUSE (FR)

(72) Inventors: Geanncarlo LUGO, Toulouse (FR); Lucie BERNARD, Toulouse (FR); Olivier NEYROLLES, CORRONSAC (FR); Muriel THOMAS, IGNY (FR); Philippe LANGELLA, VELIZY (FR); Aude REMOT, SEMBLANCAY (FR)

(21) Appl. No.: 17/593,910

(22) PCT Filed: Mar. 27, 2020
(86) PCT No.: PCT/EP2020/058832

§ 371 Date: Nov. 24, 2021

(30) Foreign Application Priority Data
Mar. 29, 2019 (FR) FR1903364

(51) Int. Cl. C12N 1/20; A61K 35/741

(52) U.S. Cl. CPC C12N 1/205; C12R 2001/225; A61K 35/741

(57) ABSTRACT

The present invention relates to the use of a bacterium of the genus Lactobacillus in the treatment of respiratory diseases in humans as well as in animals. in particular, a particular strain of this bacterium and pharmaceutical compositions comprising it are described.

The microbiota, made up of bacteria but also of viruses, parasites and fungi present in all mucous membranes, such as the intestine or the lungs, is a major component of host-pathogen interactions. The intestinal microbiota, for example, exerts a protective effect, both for the maintenance of homeostasis (tolerance mechanism), and for protection against pathogens [1]. In particular, the orientation of the immune system towards an anti-inflammatory profile, which limits inflammation, depends largely on the microbiota [2]. Its study has allowed the identification among the bacteria of the intestinal microbiota of so-called probiotic strains with specific properties for the prevention or treatment of various diseases (in particular pulmonary infections) [3, 4].

Although long considered sterile, the lungs also have a microbiota. The data available in the literature come mainly from metagenomic studies [5, 6, 7]. Indeed, the bacterial load in healthy lungs is at least an order of magnitude lower than that of the upper intestine. It has been shown that the lung microbiota consists of a relatively high diversity of bacterial species.

Colonization by the microbiota has a very important impact on immunity and health. The present inventors have thus demonstrated the protective effect of a strain of Enterococcus faecalis against allergic asthma [8]. Moreover, a probiotic candidate derived from the lung microbiota, Corynebacterium pseudodiphtheriticum, enhances the pulmonary immune response against respiratory syncytial virus (RSV) infection and pneumonia resulting from secondary infection with Streptococcus pneumoniae [9]. These issues are further developed in [10, 11].

Tuberculosis is one of the 10 leading causes of death in the world. Thanks to current treatments (Bacillus Calmette-Guérin (BCG) vaccine and four-drug therapy), the incidence of the disease is decreasing by an average of 1.5% per year. However, the appearance of antibiotic-resistant forms of tuberculosis underlines the need to identify new therapeutic strategies [12].

Tuberculosis is an infectious disease caused by a bacterium (Mycobacterium tuberculosis) and most often affecting the lungs [13]. The multiplication of the pathogen and the expression of certain molecular compounds induce immunological hypersensitivity leading to uncontrolled inflammation [14].

Once the inhaled tubercle bacillus has reached the alveoli, it is phagocyted by various immune cells, including alveolar macrophages in particular. This cellular defense is complemented by an immune defense, involving T cells through their receptors with M. tuberculosis antigens. These cells, after having multiplied locally, will migrate in the body and reach the primary infectious focus where they will trigger an inflammatory reaction.

Toxic factors secreted by immune cells, which are deleterious to the bacteria but also to host cells, lead to significant tissue damage in the lungs [15]. Thus, to effectively combat tuberculosis, it is necessary to develop new antibiotics, but also to identify tools to maintain sufficient inflammation to control the bacteria but without being deleterious to the host. This type of therapy is also important outside of tuberculosis since many respiratory diseases are linked to excessive inflammation, such as infections by Francisella tularensis or Pseudomonas aeruginosa, for example.

### DESCRIPTION

The present invention relates to new treatments of inflammation related to respiratory disease, in particular tuberculosis. In particular, the present invention relates to their prevention.

More particularly, the present inventors have shown that a bacterium of the genus Lactobacillus has very advantageous properties in the treatment and/or prevention of inflammation-related respiratory diseases such as tuberculosis.

Indeed, administration of this bacterium confers strong protection against leukocyte infiltration of the lungs, an important clinical marker of inflammation. Moreover, it leads to a strong decrease in the population of leukocytes producing pro-inflammatory cytokines in the lungs. At the same time, regulatory T cells producing anti-inflammatory cytokines are strongly stimulated. In particular, induced regulatory T cells (iTregs) are induced. Even more particularly, the iTregs that are induced are bifunctional regulatory T cells. This bacterium is unique. It has never before been described, as shown by the sequence of its 16S rRNA (SEQ ID NO: 1).

In a first aspect, the invention relates to a bacterium of the genus Lactobacillus for use in the treatment and/or prevention of inflammation associated with a respiratory disease, in particular tuberculosis. It also relates to the use of this bacterium for the preparation of a medicament for the treatment and/or prevention of inflammation related to a respiratory disease, in particular tuberculosis. Preferentially, the subject affected by the respiratory disease is a mammal, including humans, dogs, cats, equines, cattle, goats, pigs, sheep and non-human primates. More preferably, the subject is a human subject. Alternatively, the subject may be a non-human mammal, such as a dog, cat or equine.

The invention relates in particular to a particular strain of Lactobacillus for use in the treatment and/or prevention of inflammation associated with a respiratory disease, in particular tuberculosis. More specifically, the strain comprises a polynucleotide having a sequence which has at least 98% identity with the sequence of SEQ ID NO: 1. Even more specifically, the invention relates to the strain deposited under number I-5314 on Apr. 16, 2018 at the Collection Nationale des Cultures de Microorganisms (CNCM), 25 rue du Docteur Roux, 75724 Paris Cedex 15, France, for use in the treatment and/or prevention of inflammation related to a respiratory disease, in particular tuberculosis.

The invention also relates to a particular strain of Lactobacillus sp. having properties for the prevention and/or treatment of inflammation-related respiratory diseases. More specifically, the strain comprises a polynucleotide having a sequence which has at least 99% identity with the sequence SEQ ID NO: 1. This strain is preferentially a strain of Lactobacillus animalis or Lactobacillus murinus. Even more specifically, the invention relates to the strain deposited under number I-5314 on 16 Apr. 2018 at the Collection Nationale des Cultures de Microorganisms (CNCM), 25 rue du Docteur Roux, 75724 Paris Cedex 15, France.

Strain I-5314 is produced by culturing, for example, in a growth medium known to the person skilled in the art (for example, a liquid “Man, Rogosa and Sharpe” (MRS) medium) for 1 to 2 days under aerobic conditions, at a temperature of 30-37° C., with or without pH adjustment. The fermentation broth containing the bacterial cells is collected. The broth can be used as is, concentrated or freeze-dried. Advantageously, the bacteria will be collected, for example by centrifugation and then resuspended in a suitable buffer, for example phosphate-buffered saline (PBS). The bacterial concentration can be established using a flow cytometer or other equivalent process.

The strain of the invention is particularly advantageous in that it causes a strong increase in the populations of both Th17s and Tregs. The induction of Tregs is particularly important because they are primarily bifunctional Tregs which have both pro- and anti-inflammatory properties. Thus, depending on the context, biTregs can positively or negatively regulate the inflammatory response occurring during infectious disease [16, 17, 18].

According to a preferred embodiment, the invention thus relates to a bacterium of the genus Lactobacillus described above for use in the treatment and/or prevention of inflammation associated with a respiratory disease, in particular tuberculosis, the treatment and/or prevention comprising a decrease in leukocyte infiltration and an increase in pulmonary populations of Th17s as well as Tregs.

According to another preferred embodiment, the invention relates to the use of the bacterium described above for the preparation of a medicament for the treatment and/or prevention of inflammation related to a respiratory disease, in particular tuberculosis, the treatment and/or prevention comprising a decrease in leukocyte infiltration and an increase in the pulmonary populations of Th17s and Tregs.

According to an even more preferred embodiment, the Tregs are iTregs. Even more preferentially, the iTregs are bifunctional iTregs.

“T lymphocytes” or “T cells” are a type of lymphocyte (white blood cell) that play a central role in cell-mediated immunity. They can be distinguished from other lymphocytes, such as B cells and natural killer (NK) cells, by the presence of a T cell receptor (TCR) on the cell surface. As used herein, the term “T cell receptor” or “TCR” represents a receptor on the surface of T cells that is responsible for recognizing antigens bound to major histocompatibility complex (MHC) molecules. T cells do not present antigens and depend on other lymphocytes (natural killer cells, B cells, macrophages, dendritic cells) to facilitate antigen presentation. Types of T cells include in particular T helper cells (Th cells), memory T cells (Tcm, Tern or Temra), regulatory T cells (Treg), cytotoxic T cells (CTL), natural killer T cells (NKT cells), gamma delta T cells, and mucosal-associated invariant T cells (MAIT).

Among the T cells, the “CD4⁺ T cells”, also called “T helper (Th)”, have the main function of positively or negatively regulating other immune cells. These cells express the CD4 glycoprotein on their surface. The term “CD4”, as used herein, refers to a T cell membrane glycoprotein that interacts with major histocompatibility complex (MHC) class II antigens and is also a receptor for human immunodeficiency virus. The protein functions to initiate or enhance the early phase of T cell activation. Preferably, the CD4 molecule of the invention is a polypeptide having the amino acid sequence represented by NP_038516.

CD4⁺ T cells can be classified according to the type of cytokines they produce. Thus, Th1 CD4⁺ T cells, Th2 CD4⁺ T cells, Th17 CD4⁺ T cells or regulatory CD4⁺ T cells can be identified in particular.

A population of activated CD4⁺ T cells that direct the immune response toward cellular response and cytotoxicity is referred to as “CD4⁺ Th1 T cells” or “Th1 cells” or “Th1”. This primarily produce the cytokines IL-2, TNFα and IFNγ and express the transcription factor T-bet. The term “T-bet” or “TBX21”, as used herein, represents a transcription factor of the T-box family of transcription factors, which is required for the differentiation of Th1 T cells and Tc1 cytotoxic T cells (i.e., a cytotoxic T cell with receptors on its surface that can bind to complexes formed by a peptide presented by an MHC class I molecule), both of which lymphocyte populations are capable of secreting IFNγ. In a preferred embodiment, the T-bet protein has the amino acid sequence represented by NP_037483.1.

Th1 lymphocytes are induced by the cytokine IL-12 in response to infections by viral or bacterial pathogens (such as M. tuberculosis, for example). The cytokines then produced by Th1 cells activate macrophages that destroy the pathogens. However, this anti-infectious Th1 response can also be at the origin of immunopathological tissue lesions, in particular in the presence of chronic infection.

As used herein, “CD4⁺ Th17 T cells” or “Th17 lymphocyte” or “Th17 cells” or “Th17” means a population of CD4⁺ helper T cells expressing the transcription factor RAR-related orphan receptor-γt (ROR-γt) and producing the cytokine IL-17A, a pro-inflammatory cytokine. Advantageously, Th17 cells are also characterized by the release of IL-17F, IL-21 and IL-22 and the co-expression of the membrane markers CCR6, ICOS and CCR4. Th17 cells are involved in the control of extracellular bacterial and fungal infections.

As used herein, the term “regulatory T cells” or “Tregs” or “suppressor T cells” refers to a population of T cells that express the transcription factor FOXP3 and maintain immunological tolerance. Tregs cells are important in maintaining homeostasis, controlling the magnitude and duration of the inflammatory response, and preventing autoimmune and allergic responses. During an immune response, Tregs thus suppress immune reactions mediated by effector T cells, such as CD4⁺ or CD8⁺ effector T cells.

Regulatory T cells can be natural Tregs or induced Tregs. As used herein, “natural Tregs cells” or “natural Tregs” means T cells of thymic origin which express particular cell surface markers, namely CD4 and CD25 markers. Thus, the cells are preferably of CD4⁺CD25^{high}FOXP3^{high} phenotype. In addition, natural Tregs express the transcription factor Helios.

“Induced Tregs” or “induced Tregs cells” or “iTregs cells”, as used herein, are T cells of peripheral origin whose differentiation is induced as a result of antigenic interaction in the presence of cytokines such as TGF-β and IL-2. iTregs are characterized by the presence of the a chain of the IL-2 receptor (CD25) and CCR4 on their surface and the production of suppressive cytokines such as for example IL-10, in addition to the expression of FOXP3. Furthermore, iTregs cells do not express the transcription factor Helios.

The term “CD25”, as used herein, denotes the IL-2 receptor alpha chain. This protein is a type I transmembrane protein found on activated T cells, activated B cells, some thymocytes, myeloid precursors, and oligodendrocytes that associate with CD122 to form a heterodimer that can serve as a high-affinity receptor for IL-2. Tregs in particular express CD25 in addition to CD4 and FOXP3. Preferably, the CD25 molecule of the invention is a polypeptide having the amino acid sequence represented by NP_032393.

“Helios”, as used herein, is understood to mean is a zinc finger transcription factor encoded by the IKZF2 gene. The Helios transcription factor forms homodimers or even heterodimers with the Iskaros and Aiolos transcription factors. Helios is expressed in particular in Tregs cells. More specifically, Helios is expressed exclusively in natural Tregs, but not in induced Tregs. Preferentially, the Helios protein as understood herein corresponds to two isoforms, whose amino acid sequences are represented by NP_057344.2 and NP_001072994.1, respectively.

In a preferential embodiment, the iTregs cells are cells possessing dual functionality and are referred to as “bifunctional iTregs” or “bi-Tregs” or “bifunctional iTregs cells” or “bi-Tregs cells” or “biTregs”. According to this embodiment, biTregs cells are cells that express both ROR-γt and FOXP3. Preferably, the biTregs cells produce both the pro-inflammatory cytokine IL-17 and the anti-inflammatory cytokine IL-10. Even more preferably, biTregs cells additionally produce the cytokines TGF-β and IL-35.

As used herein, “RAR-related orphan receptor-γt” or “ROR-γt” means a transcription factor of the steroid hormone nuclear receptor family, exclusively expressed in cells of the immune system. The ROR-γt transcription factor thus plays a key role in regulating the differentiation of Th17 cells. Preferably, the transcription factor ROR-γt is a polypeptide having the amino acid sequence represented by NP_001001523.1.

As used herein, the term “FOXP3” refers to a transcription factor belonging to the “forkhead/winged helix” family of transcription regulators. The FOXP3 transcription factor is the master regulator of the development and function of Tregs. In addition, FOXP3 is a marker for Tregs, with expression of this transcription factor in a CD4⁺ T cell being sufficient to characterize a Treg. Preferably, the FOXP3 transcription factor is a polypeptide having the amino acid sequence represented by NP_001186276.

The present inventors have thus shown that administering the bacterium described above leads to an increase in the lung population of iTregs cells. This increase is not caused by an increase in cell proliferation, but by an induction of differentiation of these cells. Preferably, administration of the bacterium described herein leads to an induction in the lung of iTregs secreting both pro-inflammatory cytokines (for example, IL-17A) and anti-inflammatory cytokines (for example, IL-10).

The term “cytokine”, as used herein, refers to a family of small, secreted regulatory proteins that play a crucial role in immune responses. Cytokines are involved in cell-to-cell communication and regulate many cellular functions, such as cell survival and growth, as well as the induction of expression of many genes. Cytokines can be produced by many cell types. As explained above, the cell type of a given lymphocyte is determined in part by its cytokine profile. Thus, “Th1 cytokines”, as used herein, are the cytokines produced by CD4 Th1 T cells (in particular IL-2, IFNγ and TFNα).

As used herein, “pro-inflammatory cytokine” means those cytokines that lead to increased inflammation. They include in particular cytokines such as, for example, IL-1β, TNFα, IL-6, IL-15, IL-17, IFN-γ and IL-18. According to a preferred embodiment, the pro-inflammatory cytokines are TNFα, IL-6, IFN-γ and IL-17, more preferentially IL-17. The “anti-inflammatory cytokines” are those that control the pro-inflammatory cytokine response. Anti-inflammatory cytokines act in concert with specific cytokine inhibitors and soluble cytokine receptors to regulate the human immune response. The major anti-inflammatory cytokines include the IL-1 receptor antagonist, IL-10 and TGF-β. Preferentially, the anti-inflammatory cytokines are IL-10 and TGF-β.

According to a more particularly preferred embodiment, administration of the present bacterium results in the induction in the lung of iTregs producing 11-10, TGF-β and IL-17. Furthermore, the inventors have shown that the number of cells producing these cytokines is increased after administration of this bacterium. In contrast, the concentration of pro-inflammatory cytokines such as TNFα, IL-6 and IFN-γ is not affected.

The term “interleukin 17” or “IL-17” or “IL-17A”, as used herein, represents a homodimeric glycoprotein of 20-30 kDa. The human IL-17 gene encodes a protein consisting of 155 amino acids, including a 19 amino acid signal sequence and a 136 amino acid mature segment. IL-17 is a pro-inflammatory cytokine that participates in the defense against extracellular bacterial and fungal infections. Once secreted, this cytokine acts on epithelial cells, endothelial cells, fibroblasts as well as other cells of the immune system, activating them to produce pro-inflammatory cytokines such as IL-1, IL-6, TNF-α, chemokines, GM-CFS, etc.

As used herein, “interleukin 10” or “IL-10” means a homodimeric protein composed of two α-helix subunits linked by non-covalent interactions. Preferably, each IL-10 monomer is expressed as a precursor whose amino acid sequence is represented by NP_000563.1. IL-10 is a key anti-inflammatory cytokine produced by activated immune cells that plays a critical role in controlling immune responses. In particular, IL-10 reduces the expression of Th1 cytokines, MHC class II antigens and co-stimulatory molecules on macrophages. IL-10 also enhances B cell survival, proliferation and antibody production. IL-10 can block NF—KB activity and is involved in the regulation of the JAK-STAT signaling pathway.

“TGF-β” or “transforming growth factor-β” is herein understood to be a multifunctional cytokine belonging to the transforming growth factor superfamily and comprising four different isoforms (TGF-β1 to 4). Preferably, TGF-β1, 2, 3 and 4 have amino acid sequences represented by NP_000651, NP_001129071 or NP_003229, NP_001316867 or NP_001316868 or NP_003230, and Q64280.1. TGF-β is involved in multiple processes. In particular, TGF-β has an immunosuppressive and anti-inflammatory role by promoting the resolution of inflammation and the return to homeostasis. TGF-β thus suppresses the production of cytokines by inhibiting the activity of macrophages and Th1 cells. In particular, TGF-β neutralizes IL-1, IL-2, IL-6 and TNFα, and induces IL-1RA.

The term “increased”, as used herein in certain embodiments, means a larger amount, for example, a slightly larger amount than the original amount or for example, an amount in great excess of the original amount, and in particular all amounts in the range. Alternatively, “increased” may refer to an amount or activity that is at least 1%, 2%, 3%, 4%, 5%, 6%, 7%, 8%, 9%, 10%, 11%, 12%, 13%, 14%, 15%, 16%, 17%, 18%, 19%, or 20% more than the amount or activity for which the increased amount or activity is compared. The terms “increased”, “larger than”, and “increased” are used interchangeably here. Thus, an “increased lymphocyte population” means a population of the lymphocytes, for example Th17s or iTregs, in particular biTregs, increased relative to a reference control, such as, for example, a control not treated with the present bacterium. In other words, an “increased lymphocyte population” in the lungs, for example Th17 or iTregs, in particular biTregs, means that the number of the lymphocytes in the lungs is increased compared with a reference control, such as, for example, a control that has not been treated with the present bacteria. In particular, this increase may result from an increase in the differentiation of T cells into the type of lymphocytes of interest (for example Th17 cells or iTregs cells, in particular biTregs cells) and/or an increase in cell proliferation. Preferably, this increase in the population of lymphocytes of interest (for example Th17 cells or iTregs cells, in particular biTregs cells) does not result from an increase in cell proliferation.

The term “decrease”, as used herein in certain embodiments, means a smaller amount, for example, a slightly smaller amount than the original amount, or for example, a much smaller amount than the original amount, and in particular all amounts in between. Alternatively, “decreased” may refer to an amount or activity that is at least 1%, 2%, 3%, 4%, 5%, 6%, 7%, 8%, 9%, 10%, 11%, 12%, 13%, 14%, 15%, 16%, 17%, 18%, 19% or 20% less than the amount or activity for which the decreased amount or activity is being compared. The terms “decreased”, “smaller than”, “less than” and “reduced” are used interchangeably here. A “decreased lymphocyte population” thus means a reduced population of the lymphocyte compared with a reference control, such as, for example, a control not treated with the present bacterium. In other words, a “decreased lymphocyte population” in the lungs means that the number of the lymphocytes in the lungs is reduced relative to a reference control, such as, for example, a control not treated with the present bacterium.

The “control” as used herein may be a patient, an animal model or an in vitro cell model. Preferably, the “control” is a patient. As used herein, “patient” means a human subject suffering from inflammation related to a respiratory disease, in particular tuberculosis. According to another preferred embodiment, the subject is an animal, in particular a dog, a cat or a horse.

In another aspect, the present invention also relates to a pharmaceutical composition comprising the strain described herein, preferably strain I-5314, and at least one pharmaceutically acceptable excipient.

The inactivated bacterium induces the same effects as the live strain and therefore also has properties for the prevention and/or treatment of respiratory diseases.

According to a particular embodiment of the invention, the strain I-5314 present in the pharmaceutical composition is an inactivated strain. As used herein, “inactivated strain” means a bacterial strain which is unable to grow and/or divide. Preferentially, an inactivated strain no longer has any metabolic activity. However, the inactivated bacteria according to the invention are still capable of moderating inflammation, i.e., administration of the inactivated bacteria results in a decrease in leukocyte infiltration and an increase in pulmonary populations of Tregs.

Techniques for inactivating bacteria are well known to the skilled person. For example, mention may be made of heat inactivation, UV or gamma irradiation, acid treatment, hydrogen peroxide treatment, etc. The present bacteria are preferentially inactivated by heat treatment.

It is particularly advantageous to use extracts of strain I-5314 in the present pharmaceutical compositions. An “extract”, as used herein, refers to any cellular material obtained following lysis of one or more bacterial strains. Advantageously, an extract has undergone one or more additional extraction and/or purification steps. Preferentially, the extract is obtained from a single strain; more preferentially, the strain is the strain described above, in particular strain I-5314.

Lysis may be carried out by any means known to the skilled person: alkaline lysis, lysis by sonication, lysis by high pressure (French press), etc. The extract obtained by cell lysis may then be subjected to additional extraction and/or purification steps. These may comprise any treatment customary for such extracts and known to the skilled person: mention may be made, inter alia, of centrifugations (for example, to separate the plasma membrane from the cytoplasm), filtrations, precipitations and separations of the various cellular constituents (for example, using one of the many types of chromatography), etc. Each of the different extracts obtained at each of these steps can be used in the method of the invention as long as it is still capable of moderating inflammation, i.e., administration of the extract results in a decrease in leukocyte infiltration and an increase in pulmonary populations of Th17s and Tregs.

The present compositions are useful for the treatment of inflammation related to respiratory diseases.

As used herein, “respiratory disease” means diseases of the respiratory system, in particular of the lungs or bronchial tubes, or diseases that cause breathing difficulties. Many of these respiratory diseases are related to inflammation of the respiratory system, particularly the lungs or bronchi. These include, but are not limited to, asthma (mild, moderate or severe), for example, bronchial, allergic, intrinsic, extrinsic, exercise-induced, drug-induced (including aspirin and NTHEs) and dust-induced asthma, steroid-resistant asthma, bronchitis, including infectious and eosinophilic bronchitis, chronic obstructive pulmonary disease (COPD), cystic fibrosis, pulmonary fibrosis, including cryptogenic fibrosing alveolitis, idiopathic pulmonary fibrosis, idiopathic interstitial pleurisy, fibrosis complicating anti-neoplastic and chronic therapy, infection, including tuberculosis, tularemia (caused by F. tularensis), aspergillosis and other bacterial infections (for example by Francisella novicida or P. aeruginosa) or fungal infections (for example by Candida albicans or Aspergillus fumigatus); lung transplant complications; vasculitis and thrombotic disorders of the pulmonary vasculature and pulmonary hypertension (including pulmonary arterial hypertension); antitussive activity including treatment of chronic cough associated with inflammatory and secretory airway diseases and iatrogenic cough; acute and chronic rhinitis, including drug-induced rhinitis, and vasomotor rhinitis; perennial and seasonal allergic rhinitis including nervous rhinitis (hay fever); nasal polyposis; acute viral infection, including the common cold, and infection due to respiratory syncytial virus, influenza, coronavirus (including COVID-19, SARS or MRES-CoV) and adenovirus, pulmonary edema, pulmonary embolism, pneumonia, pulmonary sarcoidosis, silicosis, farmer's lung and related diseases; hypersensitivity pneumonitis, respiratory failure, acute respiratory distress syndrome, emphysema, chronic bronchitis, tuberculosis and lung cancer, etc. Respiratory diseases as understood here also include respiratory diseases specifically affecting animals, such as cats, dogs or horses. They include, in particular, kennel cough, caused in particular by Parainfluenza virus infections and Bordetella bronchiseptica bacteria. According to a preferred embodiment, the respiratory disease is tuberculosis.

As used herein, “tuberculosis” means an infectious disease caused by the bacterium Mycobacterium tuberculosis. In the vast majority of cases, tuberculosis is pulmonary tuberculosis, which means that the infection affects the lungs. When it occurs, pulmonary tuberculosis is manifested by a cough, sometimes productive or bloody, chest pain, asthenia, weight loss and night sweats. In addition, tuberculosis may be responsible for an inflammation of prolonged evolution, whose anatomopathological aspect is characteristic.

As used herein, “inflammation” means all the reactionary defense mechanisms by which the body recognizes, destroys and eliminates all substances that are foreign to it. The inflammatory reaction is the response to an insult of exogenous (infectious, traumatic) or endogenous origin (immunological cause, for example a hypersensitivity reaction or another cause, for example ischemia-reperfusion syndrome). The inflammatory response is usually composed of an initiation phase which follows an exogenous or endogenous danger signal and involving primary effectors, an amplification phase with the mobilization and activation of secondary effectors and a resolution and repair phase which tends to restore the integrity of the damaged tissue. The inflammatory reaction is thus, most often, an adapted response strictly controlled by multiple regulatory systems, including, for example, Tregs cells. However, if the inflammatory response is inappropriate or poorly controlled, it can become aggressive. In some cases, inflammation can become chronic: for example, tuberculosis leads to chronic inflammation.

As used herein, the terms “treat”, “treated”, “treatment”, and the like refer to the reduction or improvement of the symptoms of a disorder (for example, inflammation associated with a respiratory disease, such as tuberculosis) and/or the symptoms associated with it in a subject. It should be noted that, although not excluded, treatment of a disorder or condition does not require that the pathology, condition or symptoms associated with it be completely eliminated.

As used herein, the terms “prevent”, “prevention”, and the like refer to the removal of the risk of a disorder (for example, the inflammation associated with a respiratory disease, such as tuberculosis) and/or the symptoms associated therewith in a subject.

“Subject”, as used herein, is understood to mean any mammal that may benefit from the treatment described herein, including humans, dogs, cats, equines, cattle, goats, pigs, sheep and non-human primates. More specifically, a human subject is referred to herein as a “patient”. The patient may be of any age group, i.e., the patient may be a child, an adolescent or an adult. Alternatively, the subject may be a non-human mammal, such as a dog, cat or equine.

The present pharmaceutical compositions comprise, in addition to strain I-5314, one or more pharmaceutically acceptable excipients.

“Pharmaceutically acceptable excipient”, as used herein, means an excipient whose administration to an individual is not accompanied by significant deleterious effects. Pharmaceutically acceptable excipients are well known to the skilled person.

As used herein, the term “pharmaceutically acceptable excipient” includes all solvents, buffers, saline solutions, dispersion media, coatings, antibacterial and antifungal agents, isotonic and absorption retarding agents, and the like that are physiologically compatible. The excipients are selected according to the pharmaceutical form and the desired mode of administration, from the usual excipients known to the skilled person. The type of carrier will thus be chosen according to the intended route of administration. In various embodiments, the carrier is suitable for intravenous, intraperitoneal, subcutaneous, intramuscular, topical, transdermal, oral, or aerosol administration. Thus, in particular embodiments, the present strain is formulated in pharmaceutically acceptable vehicles, such as solutions, suspensions, tablets, dispersible tablets, pills, capsules, powders, sustained release formulations or elixirs, for oral administration or in sterile solutions or suspensions for parenteral administration, as well as transdermal patches and dry powder inhalers. Pharmaceutically acceptable vehicles include sterile aqueous solutions or dispersions and sterile powders for the extemporaneous preparation of sterile injectable solutions or dispersions. The use of media and agents for pharmaceutically active substances is well known in the art. Techniques and methods well known to the skilled person will thus be used to prepare pharmaceutical compositions containing strain I-5314 or extracts thereof (see for example, Ansel (1985) Introduction to Pharmaceutical Dosage Forms, 4^{th} Ed., p. 126). Processes for preparing pharmaceutical compositions, particularly pharmaceutical compositions administrable orally or by inhalation, will be known or obvious to the skilled person and are further described in, for example, “Remington's Pharmaceutical Science, 17^{th} ed., Mack Publishing Company, Easton, Pa. (1985)”, and the 18^{th} and 19^{th} editions of this manual.

The present compositions are administered to the patient at a therapeutically effective dose. The term “therapeutically effective dose”, as used herein, refers to the amount necessary to observe a therapeutic or preventive activity on inflammation related to respiratory disease, in particular tuberculosis, in particular the amount necessary to observe an improvement of symptoms. The amount of bacterium I-5314 to be administered as well as the duration of the treatment are evaluated by the skilled person according to the physiological state of the subject to be treated, as well as the administration route used. The bacterial strain used can be administered as a single dose or in multiple doses.

The skilled person will thus be able to choose the routes and modes of administration of the composition, as well as the optimal dosage and galenic forms, according to the criteria generally taken into account in the manufacture of a medicinal product or the establishment of a pharmaceutical or veterinary treatment. Preferably, these compounds will be administered systemically, in particular intravenously, intramuscularly, intradermally, intraperitoneally or subcutaneously, or orally, or topically (by means of gel, aerosols, drops, etc.). Suitable unit forms of administration include oral forms such as tablets, soft or hard capsules, powders, granules and oral solutions or suspensions, sublingual, buccal, intra-tracheal, intraocular, intranasal, inhalation, topical, transdermal, subcutaneous, intramuscular or intravenous forms of administration, rectal forms of administration and implants. For topical application, the compounds according to the invention can be used in creams, gels, ointments or lotions.

It will be particularly advantageous to administer the composition by enteral, oral, parenteral (for example subcutaneous, intradermal, or intramuscular) or mucosal (for example intranasal, sublingual, intravaginal, transcutaneous) routes. More preferably, the pharmaceutical composition will be administered on several occasions, spread out over time. Its mode of administration, dosage and optimal galenic form can be determined according to the criteria generally taken into account in establishing a treatment adapted to a patient, such as, for example, the patient's age or body weight, the severity of the patient's condition, tolerance to the treatment and the side effects observed.

In the present pharmaceutical compositions, the active ingredient(s) are generally formulated in dosage units. For example, when live I-5314 is administered, the dosage unit contains at least 10² CFU, preferentially at least 10³ CFU, preferentially at least 10⁴ CFU, preferentially at least 10⁵ CFU, preferentially at least 10⁶ CFU, more preferentially at least 10⁷ CFU, even more preferentially at least 10⁸ CFU, most preferentially at least 10⁹ CFU, per dosage unit. According to another embodiment, the dosage unit contains between 10² and 10⁹ CFU, advantageously between 10⁵ and 10⁹ CFU, preferably from 10⁷ to 10⁹ CFU per dosage unit, for daily administrations, once or more times per day. Furthermore, when bacterial extracts are administered to the patient, the dosage unit contains 2.5 to 500 mg, advantageously 10 to 250 mg, preferably 10 to 150 mg per dosage unit, for daily administrations, one or more times per day. Although these dosages are examples of average situations, there may be particular cases where higher or lower dosages are appropriate, such dosages also belong to the invention. According to the usual practice, the appropriate dosage for each patient is determined by the physician according to the mode of administration, age, weight and response of the patient.

The invention will be described more precisely by means of the following examples.

### LEGENDS OF THE FIGURES

FIG. 1: Growth kinetics of strain CNCM I-5314. Growth characteristics of lactobacilli. The growth analysis of strains CNCM I 4968, CNCM I-5314 and CNCM I 4967 was carried out by measurements on fresh cultures made from 1/100 dilutions of precultures by measurements of optical density (OD) at 600 nm and of colony forming units (CFU). These measurements allow the preparation of inocula at the desired concentration from an OD measurement using the concordance factor between OD and CFU. The representation of the LN(OD) as a function of time also makes it possible to determine the generation time of the bacteria, calculated from the slope of the curve in exponential phase. Each point represents the average of two OD measurements. A representative experiment of 2-3 independent experiments is shown.

FIG. 2: Experimental protocol used to study the effect of administration of bacteria isolated from the lung microbiota on Mycobacterium tuberculosis (Mtb) infection. Six-week-old female C57BL/6 mice were intranasally (i.n.) administered 10⁷ bacteria (CNCM I 4968, or CNCM I-5314 or CNCM I 4967 or PBS) in 25 μL of PBS 3 times a week for 2 weeks. Depending on the experiment, they are then sacrificed (FIG. 2) or infected with 10³ CFU of Mtb (other figures). For infected mice, lactobacilli administration is continued until sacrifice at a rate of twice a week (groups treated before and after infection, denoted “bef/aft”). Alternatively, lactobacilli may be administered only after infection (CNCM group I-5314 ap) and not before. After sacrifice, the lungs are used either whole for histology analysis of immunopathology or homogenized to determine bacterial load and characterize the local immune response by flow cytometry.

FIG. 3: Modification of the pulmonary immune system of uninfected mice by bacteria isolated from the lung microbiota. C57BL/6 mice are intranasally administered 10⁷ bacteria (CNCM I4968 or CNCM I-5314 or CNCM I4967 or PBS) in 25 μL of PBS 3 times a week for 2 weeks. After sacrifice, a cell suspension is obtained by enzymatic and mechanical dissociation of the lungs. The proportions of CD4⁺ T cell subpopulations are determined by flow cytometry. A. Analysis strategy. After exclusion of doublets and dead cells, CD4⁺ T cells are selected. The proportion of different subpopulations is determined by selecting cells expressing a specific intracellular factor of interest (bottom panel) and not its control isotype (top panel). B. Proportion of CD4⁺ T cells expressing Foxp3 factor (called Treg), or producing TNF-α (Th1-like cells) or IL-17 (Th17). The graphs represent the median obtained by grouping 3 experiments with 4-7 mice each. Statistic: Kruskal-Wallis test: * p<0.05; ** p<0.01; *** p<0.001; **** p<0.0001

FIG. 4: Impact of administration of bacteria isolated from the lung microbiota on Mtb infection. C57BL/6 mice are intranasally administered 10⁷ bacteria (CNCM I-4968 or CNCM I-5314 or CNCM I-4967 or PBS) in 25 μL of PBS 3 times a week for 2 weeks. They are then infected with 1000 bacteria of Mtb strain H37Rv by intranasal route. After infection, the mice are given the lung bacteria as before twice a week for 30 days. After sacrifice the lungs are dissociated to estimate the bacterial load or fixed to assess tissue damage. A. Estimation of bacterial load measured by agar medium plating of ground lung tissue. Each graph represents an independent experiment comparing 2 groups of treated mice with a control group, n=3-7 mice per group. B. Tissue damage estimated by observation of hematoxylin-eosin (HE) staining performed on histological sections of lungs fixed in 10% formalin and embedded in paraffin. Representative images from 2 independent experiments n=3 mice per group.

FIG. 5: Experimental scheme for studying cytokine production by mouse lung explants in the presence of strain CNCM I-5314.

FIG. 6: Lactate dehydrogenase (LDH) assay. Bacterium 20: CNCM 5314; bacterium 11: known cytotoxic bacterium (positive control).

FIG. 7: Characterization of the impact of lactobacillus CNCM I-5314 on cytokine secretion in mouse lung explants. Mouse lung explants are placed in the presence or absence of strain CNCM I-5314 at 37° C. After 16 hours of incubation, cytokines are assayed by the Luminex technique. A. Pro-Th1/Th1 cytokines. B. Pro-Th2/Th2 cytokines. C. Pro-Th17/Th17/Th22 cytokines. D. Th9/Treg/Prolif cytokines. E. Pro-inflammatory cytokines. Strain CNCM I-5314 is listed as bacterium 20.

FIG. 8: Characterization of the impact of Lactobacillus I-5314 administration on Mtb infection. C57BL/6 mice receive PBS (white bars) or 10⁷ of lactobacillus intranasally before and after infection (CNCM I5314 bef/aft group, gray bars) per 1000 CFU of Mtb strain H37Rv intranasally, or only after infection (CNCM I-5314 aft group, hatched bars). A. Estimated bacterial load 42 days post-infection, measured by agar media plating of ground lung tissue. The graph shows a representative experiment from 2 independent experiments, n=6 mice per group. B. Tissue damage estimated by observation (left panel) and quantification of leukocyte infiltrates (right panel) on hematoxylin-eosin stains performed on histological sections of lungs fixed in 10% formalin and embedded in paraffin 42 days after infection. Percentage infiltration is the ratio of the area occupied by leukocyte infiltrates to the total area of the lungs. 2-5 experiments with 4-5 mice per group are shown. C.D. Characterization of lung CD4⁺ T cells present 42 days after infection by flow cytometry. The proportions of CD4⁺ TC expressing different transcription factors (C.) (T-bet, characteristic of Th1, RORγt for Th17 and Foxp3 for Treg) or producing cytokines (D.) after stimulation with phorbol 12-myristate 13-acetate (PMA) and ionomycin in the presence of monensin and brefeldin A (IFN-γ and TNF-α for Th1, IL-17 for Th17, IL-10 and TGF-β for Treg) are shown. E.F.G. Characterization of Tregs expressing Foxp3. The origin of Tregs, natural (nTreg, expressing Helios factor, top) or induced (iTreg, not expressing it, bottom) (E.), their proliferation (characterized by Ki67 antigen expression) (F.) and cytokine production (G.) is detected by flow cytometry as in C and D. Statistics: graphs corresponding to I-4 independent pooled experiments with 4-7 mice per group for panels C and D. The median of each group is shown and a Kruskal-Wallis test compares treated mice with control mice: * p<0.05; ** p<0.01; **** p<0.0001.

### EXAMPLES

### Materials and Methods

### Bacterial Strains, Media, Growth Conditions

The pulmonary bacterial strains were isolated from mouse lung homogenates with a homogenizer (ULTRA-TURRAX (IKA) or TissueLyser (Qiagen)). They were then cultured on yhBHI, M17, MRS, or Mannitol Salt Agar (BD biosciences) for 24 to 48 h at 37° C. under aerobic conditions or 5 days at 37° C. in a Freter chamber under anaerobic conditions. Isolated strains were frozen at −80° C. in 16% glycerol. The identity of each strain was confirmed by mass spectrophotometry and PCR sequencing of 16S RNA. The selected strains were deposited at the Collection Nationale des Cultures de Microorganisms (CNCM). The three strains used here are lactobacilli strains deposited under the references CNCM I-5314, (CNCM I-4967) and (CNCM I-4968). These bacteria are cultured in MRS liquid medium for 24 h at 30° C. (pre-culture) or 3-4 h at 37° C. (for instillations) without agitation.

### Material and Method for Sequencing

The bacterial pellet from a 15 mL culture of strain I-5314 (exponential phase) was centrifuged (1550 g, 5 min) and frozen at −20° C. This bacterial pellet was taken up in saline solution (30 mM NaCl, 2 mM EDTA (pH=8.0) then centrifuged and resuspended in lysis buffer (20 mM Tris HCl pH 8.0, 2 mM EDTA, 1.2% Triton® X-100) enriched in lysozyme (20 mg/mL, Sigma-Aldrich #L6876) for 2 h at 37° C. Proteinase K (DNeasy® Blood Et Tissue kit) and RNase A were added for an additional 1 h at 55° C. The same volume of buffer “AL” (DNeasy® Blood Et Tissue kit) was added to the lysate, vortexed and incubated 30 min at 56° C. After incubation, the same volume of 100% ethanol was added. DNA was extracted using the DNeasy Mini spin columns kit (Qiagen) and following the vendor's instructions.

### DNA Sequencing.

Sequencing was produced by the “GeT-PlaGe” platform (INRA, Castanet-Tolosan, France). The DNA was fragmented by sonication to obtain fragments of 200 to 1000 base pairs (bp). These fragments were added to Illumina adapters and sequenced using the “Illumina HiSeq 3000” method.

### Genome Assembly and Annotation.

The raw sequences were put in fastp, version 0.19.4 format to remove Illumina-like adaptor sequences and low-quality sequences. The sequences were assembled by “Unicycler version, v0.4.7” and the quality of the assembly was verified by QUAST v5.0.2, b7350347c. The assembled genome is visualized by “Bandage v0.8.1” and annotated by Prokka v1.13.

### Bacterial Growth Kinetics

The growth profile was determined by spectrophotometry (Spectronic instruments 20; Genesys) which allows the measurement of the optical density (OD) from the culture medium containing the bacteria. To this end, the bacteria were pre-cultured on agar plates and then subcultured in 10 mL of Hiveg BHI medium (bacterium 20=CNCM I-5314). Growth was monitored by measuring the OD at 600 nm of the bacterial culture every hour from 0 h to 9 h+a reading at 24 h.

### Murine Model of Tuberculosis Infection and Probiotic Treatment

All experiments performed on animals were approved by the French Ministry of Higher Education and Research after review by the Regional Ethics Committee (APAFIS Approval 5704). The mice used were C57BL/6 6-to-8-week-old females from Charles River Laboratories.

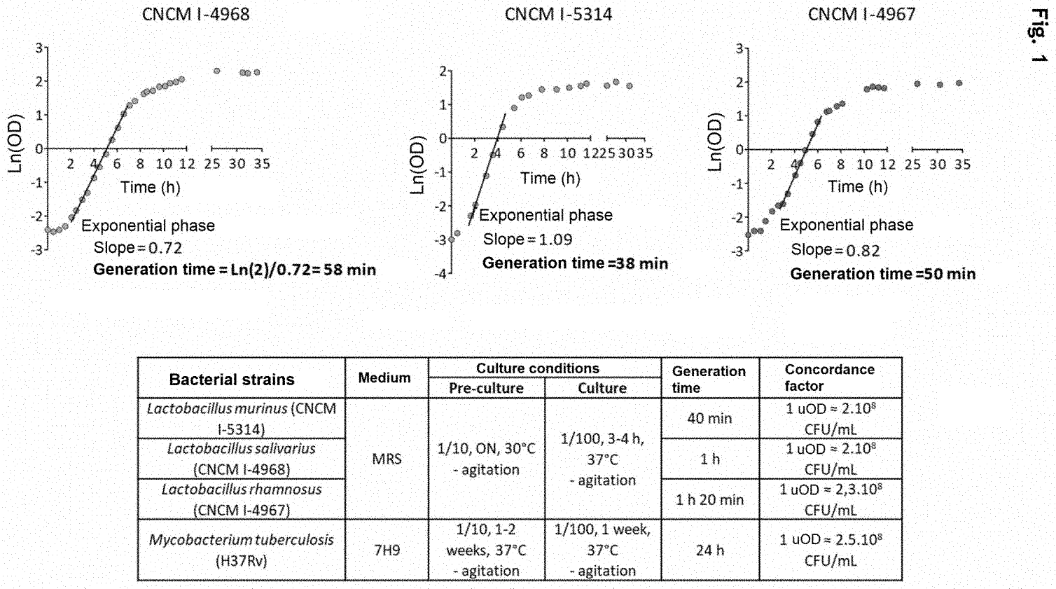

For each in vivo administration of one of the lactobacilli, a bacterial suspension containing 4.0×10⁸ CFU/mL was prepared in phosphate-buffered saline (PBS) from fresh cultures in exponential phase. Mice received 25 μL of PBS containing 1.0×10⁷ CFU or 25 μL of PBS (control group) intranasally (i.n.) under gas anesthesia (4% isoflurane, Virbac Denmark). This is repeated 3 times per week for 2 weeks and then the mice are either sacrificed (experiments on uninfected mice) or infected (procedures described below), and receive commensal bacteria administration again twice per week until sacrifice. In certain experiments, lactobacilli are administered only after infection and not before and after infection (see FIG. 1).

A fresh culture of Mtb strain H37Rv (grown in 7H9 liquid medium (Difco) supplemented with 0.5% glycerol, 10% ADC (Middlebrook) and 0.05% tyloxapol) is used to infect the mice. Each mouse receives i.n. 1.0×10³ CFU of Mtb in 25 μL of PBS under isoflurane anesthesia. Mice are sacrificed by cervical dislocation (under isoflurane anesthesia) after 42 days of infection.

### Histological Analysis

Whole lungs from mice dedicated to histological analysis are used. They are inflated and fixed for 5 days at 4° C. with a solution containing 10% formalin (Formalin solution, Sigma-Aldrich) and embedded in paraffin. Hematoxylin-eosin (HE) staining was performed to visualize leukocyte infiltrates, which were quantified, after scanning, with the CaseViewer software (3DHISTECH).

### Preparation of Lung Homogenates and Determination of Bacterial Load

Whole lungs from mice were collected in a sterile manner, homogenized with a gentleMACS dissociator before (C-tubes, cycle m_lung_01, Miltenyi) and after (cycle m_lung_02) 30 min incubation at 37° C. with collagenase D (2 mg/mL, Roche) and DNase I (0.1 mg/mL, Roche). A portion of this homogenate is serially diluted in PBS and then spread on 7H10 agar medium (Difco) supplemented with peptone and OADC (Middlebrook). After 2-3 weeks, the count of Mtb colonies obtained allows the estimation of the lung bacterial load. The remaining homogenates were passed through a 70 μm filter to destroy aggregates, and centrifuged at 329×g for 5 min. The supernatants were passed twice through 0.2 μm filters and stored at −80° C. for analysis of cytokines present in the lungs. Red blood cells in the pellet are lysed for 5 min with a solution containing 150 mM NH₄Cl, 10 mM KHCO₃, 0.1 mM EDTA (pH 7.2), neutralized by addition of RPMI medium containing 10% fetal calf serum (FCS). The cell suspension thus obtained is filtered through a 40 μm filter (removal of lysed red cell aggregates) for flow cytometry analysis.

### Flow Cytometry

Analysis of the different CD4⁺ T helper populations is performed by flow cytometric detection of transcription factors and cytokines characteristic of these subpopulations by labelling the cells present in the cell suspension obtained as described in the previous section. A portion of the cell suspension is incubated in RPMI containing 50 ng/mL phorbol myristate acetate (PMA, Sigma Aldrich) and 500 ng/mL ionomycin (Sigma-Aldrich) to induce cytokine production by lymphocytes as well as brefeldin A (Golgi plug 1/1000, BD Biosciences) and monensin (Golgi Stop 1/2000, BD Biosciences) to segregate these cytokines in the Golgi apparatus for 4 h at 37° C. and 5% CO₂. The rest of the cell suspension, used for transcription factor labelling, is kept in cell staining buffer (CSB, Biolegend) for 4 h at 4° C. Extracellular labeling of these two fractions is performed in CSB for 30 min at 4° C. in the dark using an anti-cluster differentiation 16/32 antibody (CD16/CD32, Biolegend) to limit aspecific labeling, a viability marker (live/dead fixable blue dead cell stain kit, Invitrogen), an anti-CD452 BV711 (clone 104, BD Biosciences), an anti-CD3 FITC (17A2, Biolegend) or anti-T cell receptor beta (TCRb) Alexa 700 (H57-597, Biolegend) and an anti-CD4 BV786 (Sk3, BD Biosciences). For intracellular labeling, cells are then fixed for 30 min at room temperature (RT), permeabilized for 15 min at RT (Foxp3/transcription factor staining buffer set, eBioscience) and incubated for 45 min at RT with a panel of antibodies including an anti-RORgt PE-CF594 (Q31-378, BD Biosciences) anti-T-bet PE-Cy7 (eBio4BIO, eBiosciences), anti-Foxp3 APC (FJK-16s, eBioscience), anti-Helios APC-eFluor 780 (22F6, eBiosciences), anti-Ki67 Alexa 700 (SolA15, eBioscience) or an anti-interleukin 10 (IL-10) FITC (JES5-16E3, BD Biosciences), an anti-IL-17 PE (TC11-18H10, BD Biosciences), an anti-IFNγ PE-Dazzle (XMG1.2, Biolegend), an anti-TNFα PE Cy7 (MP6-XT22, BD Biosciences), the anti-Foxp3 APC and an anti-TGF-β BV421 (TW7-16B4, BD Biosciences). For experiments performed on Mtb-infected mice, cells are fixed for 2 h in 4% paraformaldehyde (PFA) at RT. Data are acquired with a FACS LSRII or Fortessa (BD Biosciences) and analyzed on FlowJo V10 software. Doublets (FSH-H vs. FSC-W and SSC-H vs. SSC-W) and dead cells (live/dead positive) are excluded at the beginning of each analysis.

### Statistical Analysis

The statistical analysis of the results was performed with GraphPad Prism 7 software. On the graphs, each point represents a different mouse. The median of each group is represented by the bars. A Mann-Whitney test (comparison of 2 groups) or a Kruskal-Wallis test (comparison of 3 groups) was used to compare the values. Significance is represented by: * p<0.05; ** p<0.01; *** p<0.001; and **** p<0.0001.

### Precision-Cut Lung Slices

Precision-cut lung slices (PCLS) were obtained from fresh lungs using a Krumdieck Md. 6000 microtome (Alabama Research and Development, Munford, Ala., USA). The lungs were filled with 1.5% low-melting agarose in RPMI (Invitrogen, Villebon sur Yvette, France) heated to 37° C. via the trachea. After 1 min for solidification, the lungs were placed in the Krumdieck microtome chamber filled with cold PBS and cut to a thickness of 200 μm. Two of the PCLS per well were then placed at 37° C., 5% CO₂, in P24 well plates (Nunc, Sigma-Aldrich, Lyon, France) with 1 mL of RPMI 1640 (Gibco, Sigma-Aldrich, Lyon, France) supplemented with 10% heat-inactivated fetal calf serum (Gibco) and 2 mM L-glutamine (Gibco). The medium was changed every 30 min for 2 h to remove agarose, as well as one last time after overnight incubation. PCLS were then co-incubated for 24 h with lung bacteria.

### Assay of Lactate Dehydrogenase

The lactate dehydrogenase (LDH) assay is used to determine the potential cytotoxicity of bacteria on lung explants. Indeed, the release of LDH is associated with cell death. The LDH assay was performed on explant lysates and on supernatants recovered 16 h post-culture with bacteria. For this assay, LDH substrate (Promega) is added to each well containing either explant lysates or post-culture supernatants. Lysis buffer is used as a blank for the explant lysates. Incubation is done at room temperature in the dark for 30 to 40 minutes. The reaction is then stopped with 100 μL of stop solution, the plate is then read at 490 nm with a plate reader (TECAN Infinite M200 pro) using the “I-control” software.

The assay of LDH on explant lysates and supernatants is used to determine the cytotoxicity of bacteria on lung explants. For LDH assayed on explant lysates, the blank must be subtracted from the values obtained.

Cytotoxicity represents the percentage ratio between the LDH released, i.e., present in the lung explant supernatant, and the total LDH present (in the supernatants+in the lung explant lysates), represented by the following calculation

$$Cytoxicity ( \% ) = \frac{ODLDH released ( supernatants )}{\begin{matrix}ODLDH released ( supernatants ) + \\ ( ODLDH ( explant lysates ) - blank )\end{matrix}} \times 100$$

### Cytokine Assay

Cytokines were assayed using the Luminex technique. The Luminex technique uses magnetic beads with their own fluorescence, which allows a large number of cytokines to be assayed at the same time. Here, the magnetic bead has anti-IL-2 capture antibodies. Thus, when the supernatants are brought into contact with the beads in the wells, this bead will bind specifically to IL-2 via its multiple capture antibodies present on its surface. This identifies the cytokine bound to the bead. The detection antibody also recognizes the cytokine but is bound to the streptavidin-PE. The concentration of the assayed sample is directly proportional to the fluorescence intensity of the PE.

Cytokines were determined from the supernatant recovered 16 h post-culture with the bacteria. We used a Luminex kit to determine the concentration of fifteen cytokines in a single assay (Thermofisher).

TABLE 1
Cytokines measured. The cytokines assayed correspond to the
cytokines released during different immune responses such as
the type 1, 2, 9 or 17 and 22 response.

| | Pro- | Th1/Pro | | Th17/ | | Cell |
|---|---|---|---|---|---|---|
| Immunity | inflammatory | Th1 | Th2 | Th22 | Th9 | proliferation |
| Cytokines | GM-CSF | IFN-γ | IL-4 | IL-17A | IL-9 | IL-2 |
| | IL-1β | IL-12p70 | IL-5 | IL-22 | | |
| | TNF-α | IL-18 | IL-10 | | | |
| | IL-6 | | IL-13 | | | |

The plate is read on the Luminex Magpix using the “Xponent” software and then analyzed using the “Bioplex Manager” software (Biorad version 6).

### Results

The identification of bacteria from the lung microbiota capable of modifying the immune response during Mtb infection is based on a bank of primary colonizing bacteria from the lung of mice isolated and identified by Dr Langella's team (Probihôte—MICALIS—INRA), and in particular by Aude Remot and Muriel Thomas. One of these bacteria, a strain of Enterococcus sp. deposited at the CNCM under the number I-4969 is able to modulate the susceptibility of mice to allergic asthma (see WO 2017/12987 and [8]). Among these bacteria, 3 are lactobacilli that are Generally Recognized As Safe (GRAS) and have been deposited at the CNCM under the numbers: CNCM I-4968, CNCM I-5314 and CNCM I-4967, respectively.

The genome of the strain deposited under number I-5314 has been sequenced. Using sequence homology analysis software (Blast), it was found that the DNA sequence encoding the 16S rRNA (SEQ ID NO: 1) has more than 98% homology with reference strains of L. animalis and strains of L. murinus.

The growth profile of each of the three strains, CNCM I-4968, CNCM I-5314 and CNCM I-4967, was determined by measuring the OD at 600 nm. It is presented in FIG. 1.

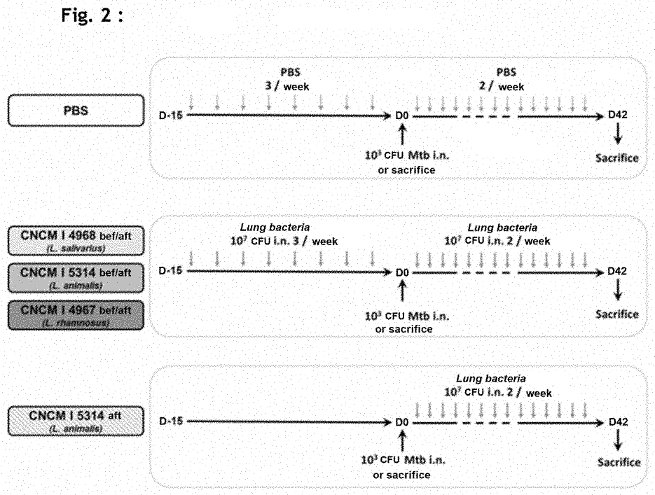

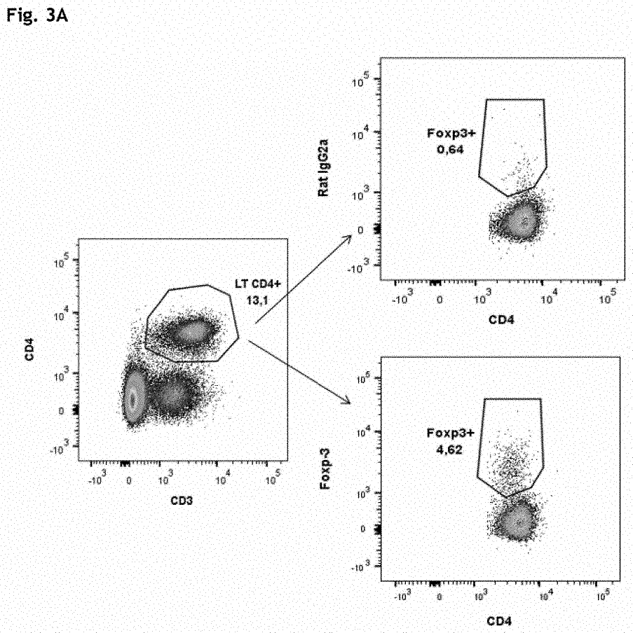

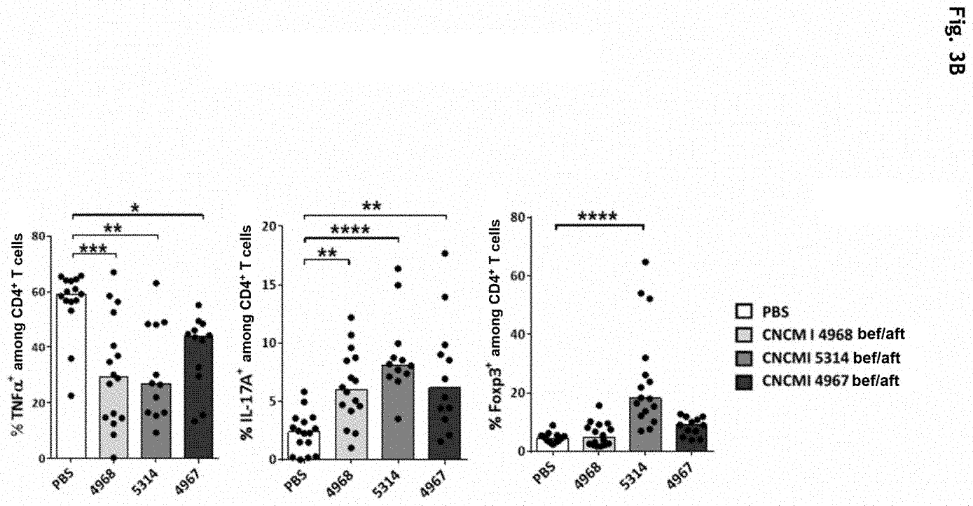

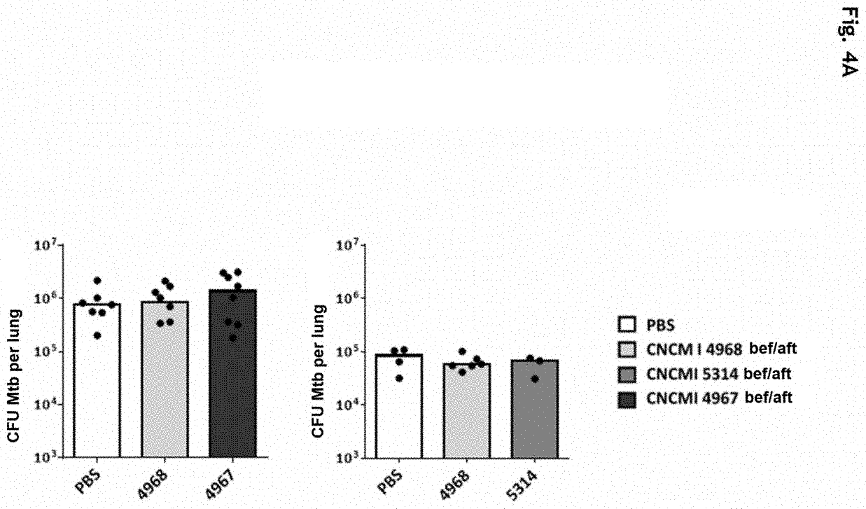

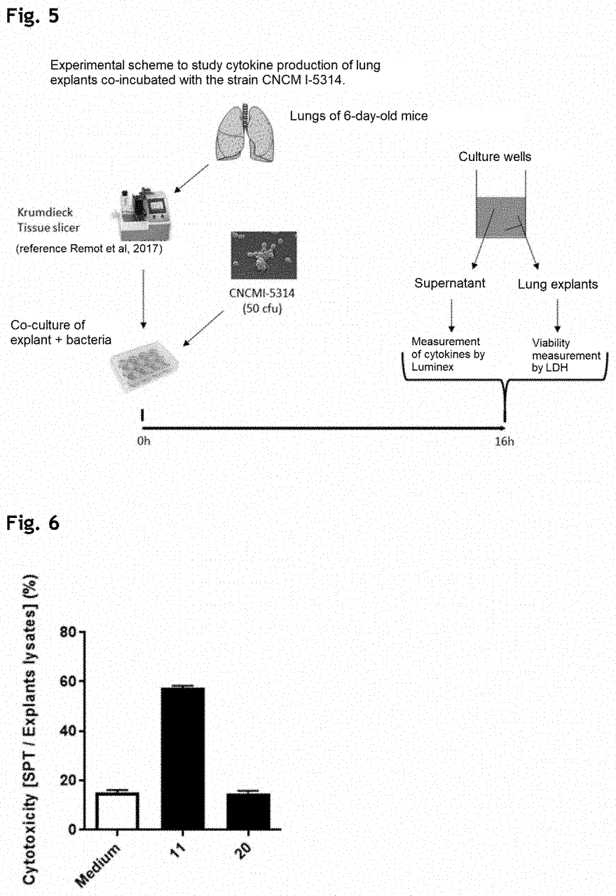

To determine the probiotic potential of these 3 lactobacilli for the prevention and treatment of tuberculosis, different protocols summarized in FIG. 2 were used in a mouse model. The administration of 10⁷ bacteria is performed intranasally for two weeks before the mice are sacrificed (FIG. 3) or infected with Mycobacterium tuberculosis (Mtb) (other figures). In these experiments the bacteria are administered before and after infection (bef/aft groups) or only after infection (aft group, FIG. 5).

As a first step, the ability of bacteria to modify the local immune system of uninfected mice was assessed by flow cytometric analysis of the expression of intracellular markers characteristic of different subpopulations of CD4⁺ helper T cells (analysis strategy in FIG. 3A), key orchestrators of the anti-Mtb immune response: Th1 (TNF-α producing), Th17 (IL-17A producing) and regulatory T cells (Treg expressing Foxp3) [16, 17, 18]. Our results show that the three lactobacilli isolated from the lung microbiota have strong immunomodulatory capabilities, with an anti-inflammatory profile (even outside an infectious context). Strain CNCM I-5314 is that which induces the strongest decrease in Th1, increase in Th17 and pulmonary Treg (FIG. 3B), while these three species belong to the same bacterial genus.

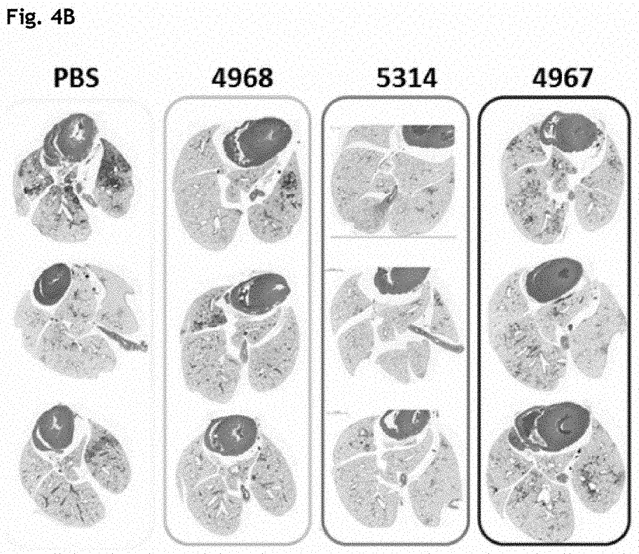

To determine whether these bacteria (and in particular strain CNCM I-5314) induce a sufficient anti-inflammatory response to decrease the immunopathology associated with tuberculosis, the bacteria were administered as before intranasally, 15 days prior to infection with Mtb and then throughout the infection (FIG. 2). In this model, the three bacteria did not alter the bacterial load of Mtb (FIG. 4A). In contrast, CNCM I-5314 appears to confer significant protection against leukocyte infiltration of the lungs (less lung surface area occupied by immune cells) leading to immunopathology in the late stages of infection (and the other two bacteria do not) (FIG. 4B).

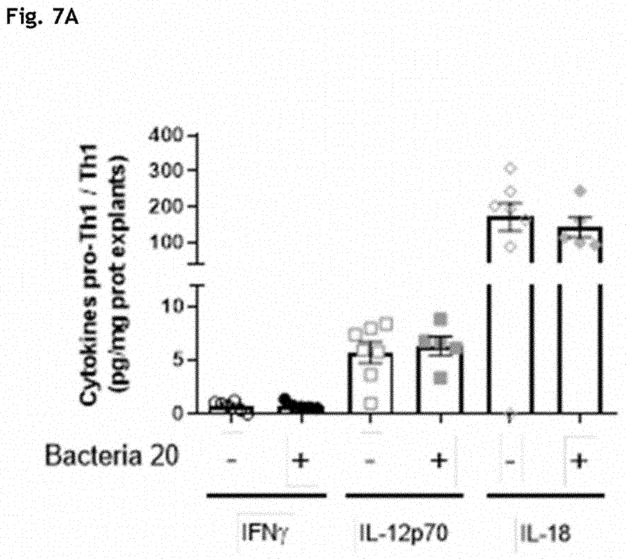

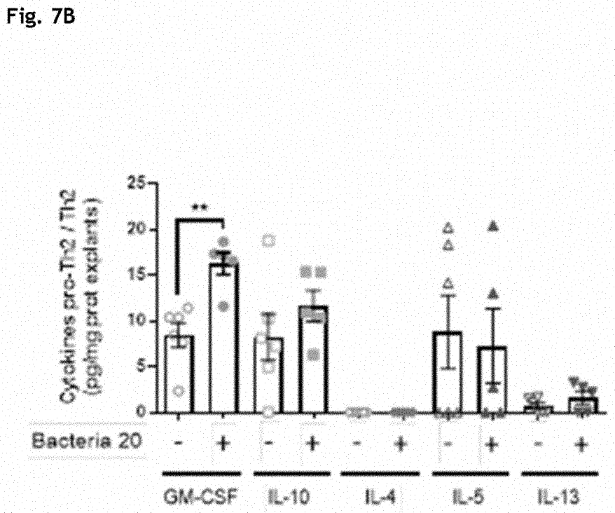

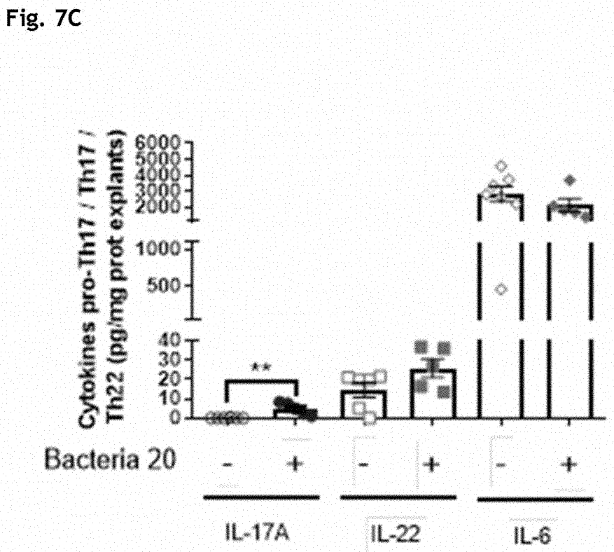

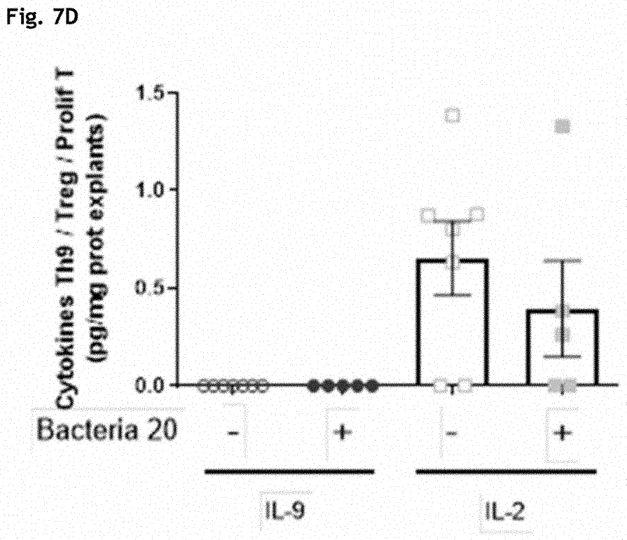

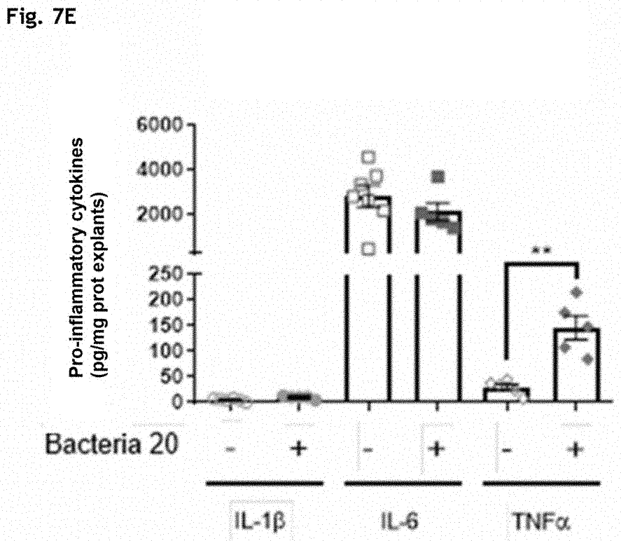

Cytokine production induced by strain CNCM I-5314 was determined according to the experimental scheme in FIG. 5 (see also Remot et al., 2017). Precision-cut slices of 6-day-old mouse lung were cultured in the presence or absence of CNCM I-5314. After 16 hours of culture, secreted cytokines were assayed in the medium, while cell viability was assessed by LDH assay (FIG. 6). The LDH assay showed no decrease in lung explant viability, indicating that strain CNCM I-5314 is not toxic (unlike a control strain). In particular, the cytokine assay showed induction of GM-CSF, IL-17a and TNFα(FIG. 7). These data made it possible to establish the immunomodulatory profile of strain CNCM I-5314 with respect to mouse lung explants.

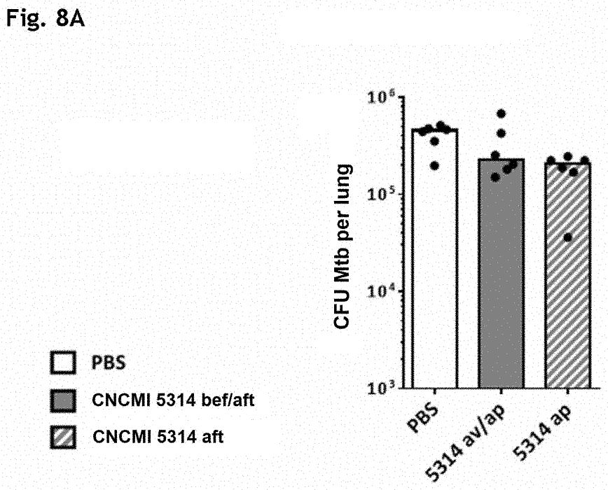

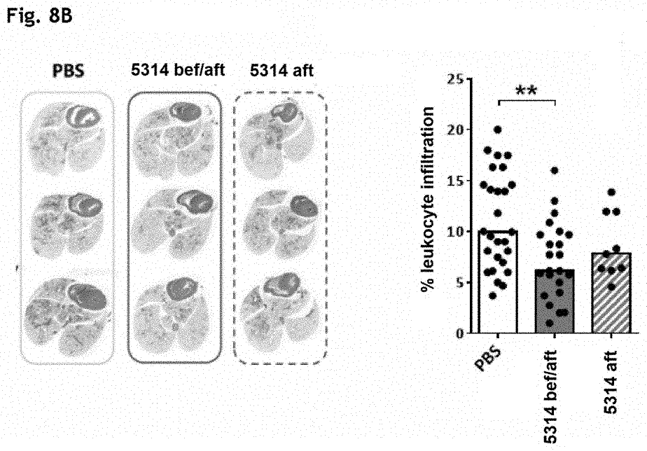

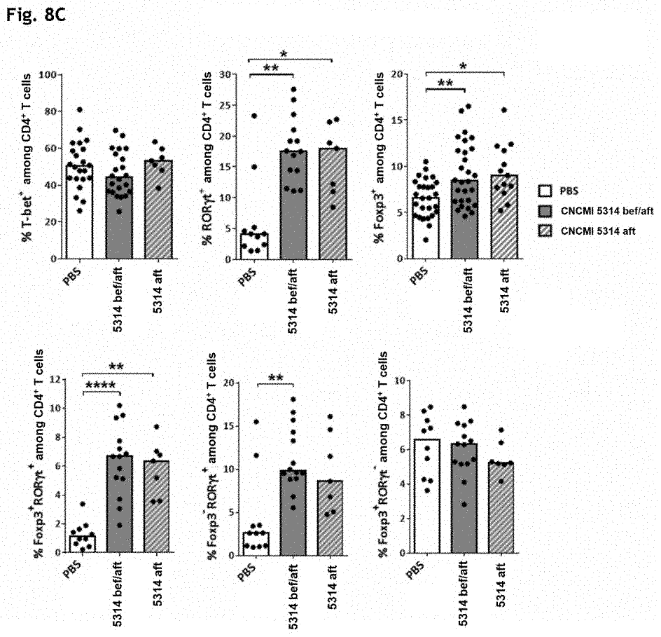

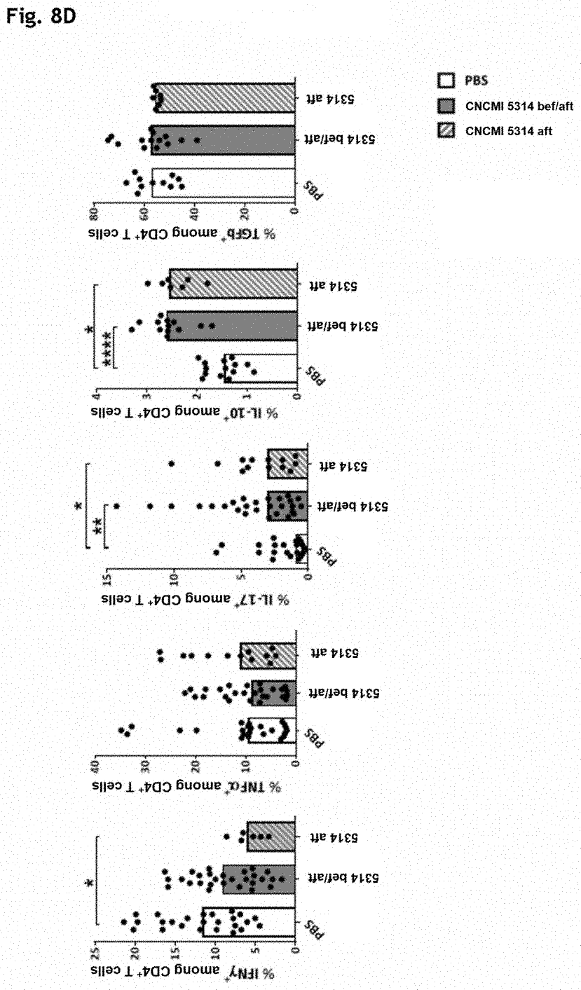

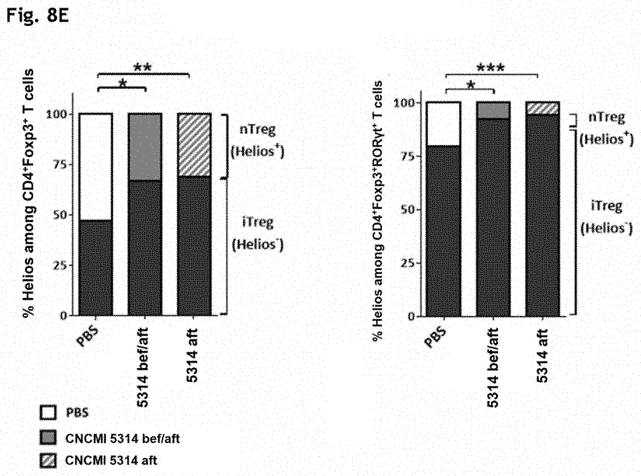

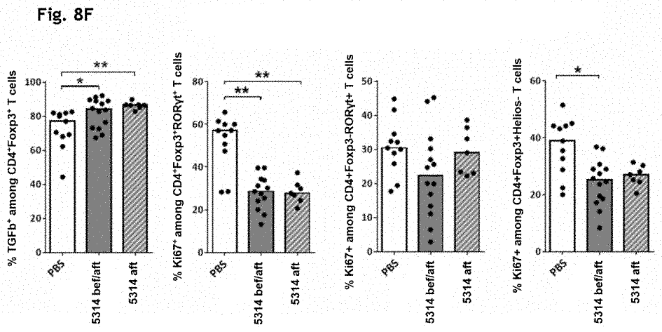

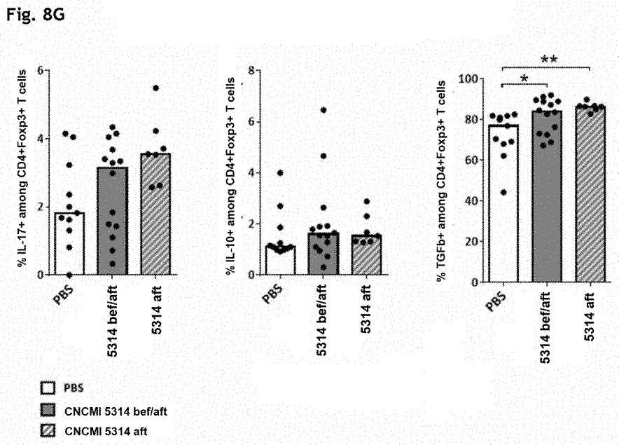

To better characterize the protective effect of strain CNCM I-5314, the same experiment was repeated (CNCM I-5314 bef/aft group) including analyses to determine the composition of the lung immune infiltrate. In addition, the capacity of this bacterium to exert its protective effect in a treatment strategy (as opposed to the prophylactic approach described above) was evaluated by adding a group for which the administration of the bacterium began only after infection (CNCMI 5314 aft group) (details of the different groups in FIG. 2). These experiments confirm that the anti-inflammatory effect induced by this bacterium does not allow Mtb to multiply uncontrollably (FIG. 8A) while it grants a significant protection (for the group having received the bacterium in pre-treatment, CNCM I-5314 bef/aft) against the immunopathology induced by the infection with a reduction of the leukocytic infiltrates (FIG. 8B). Although lung infiltration of pro-inflammatory Th1 lymphocytes (expressing T-bet and producing TNF-α or IFN-γ) varies little between groups, strain CNCM I-5314 induces an increase in both Th17 (expressing RORγt and producing IL-17A) and Treg (expressing Foxp3, producing IL-10 and TGF-β) lymphocytes at 42 days post-infection (FIG. 8C). However, further analysis reveals that the increase in Tregs observed with administration of the bacterial strain (whether with a prophylactic or treatment approach) is not related to an increase in conventional Tregs (Foxp3⁺RORγt⁻), but to that of a recently described, double-positive Foxp3⁺RORγt⁺ population called bi-Treg. These cells possess both pro- and anti-inflammatory functions and produce IL-17 but also IL-10, TGF-β and IL-35 (not analyzed here) which make them key regulators of inflammation [17, 18]. They could therefore be responsible for the decrease in leukocyte infiltrates (observed in FIG. 8B) and thus the promising effect of this bacterium for the prevention and treatment of tuberculosis. The proportion of Th17 (Foxp3⁻RORγt⁺) also appears to be increased. In the case of an environment rich in IL-10 and TGF-β (as is the case here), Th17/IL-17 activity is biased and these cells exert anti-inflammatory and protective functions with respect to tissue damage, mainly via IL-22 production (production not measured in our experiments), suggesting a beneficial role of these cells in our context [16, 17]. Tregs enhanced by CNCM I-5314, and in particular biTregs, do not express the transcription factor Helios, indicating that they are induced in the mucous membranes (iTreg) as opposed to naturally occurring Tregs (nTreg), i.e., generated in the thymus, suggesting that this effect is specific and related to peripheral tolerance (FIG. 8E). The increase in these cells does not appear to be related to increased proliferation since Ki67 antigen expression is decreased (FIG. 8F). They seem to be able to have the pro- and anti-inflammatory effects described in the literature since the Tregs observed produce IL-17, IL-10 but above all TGF-β (the production of which is also higher in the two groups of mice having received the administration of this bacterial strain) (FIG. 8G).

We show here for the first time that a bacterial strain from the lung microbiota (CNCM I-5314) is capable of modifying the immune response to Mtb, in particular through the induction of biTregs that could better control the immune balance (pro-/anti-inflammatory) and thus reduce the immunopathology induced by the infection. These results thus present the present strain of Lactobacillus (CNCM I-5314) isolated from the pulmonary microbiota as a promising probiotic for tuberculosis. Preliminary results obtained with the “CNCM I-5314 aft” group also suggest applications for the treatment of this disease. As other respiratory diseases are related to inflammation, we assume that its beneficial role would not be limited to tuberculosis.

### REFERENCES

1: Belkaid Y, Hand T W. Role of the microbiota in immunity and inflammation. Cell. 2014; 157; 121-41.
2: Atarashi K et al. Induction of colonic regulatory T cells by indigenous Clostridium species. Science (2011) 331(6015):337-41
3: Patel R, DuPont H L. New approaches for bacteriotherapy: prebiotics, new-generation probiotics, and synbiotics. Clin Infect Dis. 2015; 60; S108-21.
4: Vieira A T, et al. Control of Klebsiella pneumoniae pulmonary infection and immunomodulation by oral treatment with the commensal probiotic Bifidobacterium
5: Sibley C D, Grinwis M E, Field T R, Eshaghurshan C S, Faria M M, et al. (2011) Culture Enriched Molecular Profiling of the Cystic Fibrosis Airway Microbiome. PLoSONE 6: e22702.
6: Gollwitzer E, Saglani S, Trompette A, Yadava K, Sherburn R, et al. (2014) Lung microbiota promotes tolerance to allergens in neonates via PD-L1. Nature medicine 20: 642-647.
7: Erb-Downward J R, Thompson D L, Han M K, Freeman C M, McCloskey L, et al. (2011) Analysis of the Lung Microbiome in the “Healthy” Smoker and in COPD. PloSONE 6.
8: Remot A, et al. Bacteria isolated from lung modulate asthma susceptibility in mice. ISME J. 2017; 11; 1061-1074.
9: Kanmani P, et al. Respiratory Commensal Bacteria Corynebacterium pseudodiphtheriticum Improves Resistance of Infant Mice to Respiratory Syncytial Virus and Streptococcus pneumoniae Superinfection. Front Microbiol. 2017; 8; 1613.
10: Dumas A, et al. The role of the lung microbiota and the gut-lung axis in respiratory infectious diseases. Cell Microbiol. 2018; e12966.
11: Mathieu E, et al. Paradigms of lung microbiota functions in health and disease, particularly, in asthma. Frontiers in Physiology 2018; 9; 1168.
12: OMS 2016
13: Pai M, Behr M A, et al. Tuberculosis. Nat Rev Dis Primers. 2016; 2; 16076.
14: Nunes-Alves C, Booty M G, Carpenter S M, Jayaraman P, Rothchild A C, Behar S M. In search of a new paradigm for protective immunity to T B. Nat Rev Microbiol. 2014; 12; 289-99.
15: Malherbe S T, et al. Persisting positron emission tomography lesion activity and Mycobacterium tuberculosis mRNA after tuberculosis cure. Nat Med. 2016; 22; 1094-1100.
16: Sehrawat S, et al. Interplay of Regulatory T Cell and Th17 Cells during Infectious Diseases in Humans and Animals. Front Immunol. 2017; doi: 10.3389/fimmu.2017.00341
17: Kluger M A, et al. RORγt(+)Foxp3(+) Cells are an Independent Bifunctional Regulatory T Cell Lineage and Mediate Crescentic G N. J Am Soc Nephrol. 2016; doi: 10.1681/ASN.2014090880. Epub Jun. 8, 2015.
18: Krebs C F, Steinmetz O M. CD4⁺ T Cell Fate in Glomerulonephritis: A Tale of Th1, Th17, and Novel Treg Subtypes. Mediators Inflamm. 2016; 2016:5393894. Epub Nov. 15, 2016.

1. A bacterium of the genus Lactobacillus for use in the treatment and/or prevention of inflammation associated with a respiratory disease, the bacterium comprising a polynucleotide having a sequence which has at least 98% identity with the sequence of SEQ ID NO: 1.

2. The bacterium for use as claimed in claim 1, characterized in that the bacterium is a Lactobacillus animalis or a Lactobacillus murinus.

3. The bacterium for use as claimed in any one of claim 1 or 2, characterized in that the bacterium is inactivated.

4. The bacterium for use as claimed in any one of claims 1 to 3, characterized in that the treatment and/or prevention comprises a decrease in leukocyte infiltration and an increase in pulmonary populations of Th17 cells as well as Tregs cells.

5. The bacterium for use as claimed in claim 4, characterized in that the Tregs are iTregs.

6. The bacterium for use as claimed in claim 5, characterized in that the iTregs are biTregs.

7. The bacterium for use as claimed in any one of claims 1 to 6, characterized in that the respiratory disease is tuberculosis.

8. The bacterium for use as claimed in any one of claims 1 to 6, characterized in that the bacterium is the strain Lactobacillus sp. deposited at the Collection nationale des cultures de microorganisms (CNCM, 25 rue du Docteur Roux, 75724 Paris Cedex 15, France) under the number I-5314.

9. A strain of Lactobacillus sp. deposited at the Collection nationale des cultures de microorganisms (CNCM, 25 rue du Docteur Roux, 75724 Paris Cedex 15, France) under number I-5314.

10. A pharmaceutical composition comprising the strain of claim 9 and at least one pharmaceutically acceptable excipient.

11. The composition of claim 10 characterized in that the strain is inactivated.

12. The composition of claim 11 characterized in that the strain is heat inactivated.

13. The composition of claim 11 characterized in that the strain is present in the form of extracts.
